(19) United States
(12) Patent Application Publication
(10) Pub. No.: US 2025/0017676 A1
(43) Pub. Date: Jan. 16, 2025

(54) ROBOTIC UNIT FOR MICROSURGICAL PROCEDURES

(71) Applicant: ForSight Robotics Ltd., Yokneam Illit (IL)

(72) Inventors: Tal Korman, Tel Aviv (IL); Ariel Gil, Haifa (IL); Yoav Golan, Tel Aviv (IL); Ofer Arnold, Ma'ale Zvia (IL); Daniel Glozman, Kfar Yona (IL)

(21) Appl. No.: 18/714,027

(22) PCT Filed: Dec. 1, 2022
(86) PCT No.: PCT/IB2022/061635

§ 371 Date: May 28, 2024

Related U.S. Application Data
(60) Provisional application No. 63/285,218, filed on Dec. 2, 2021.

(51) Int. Cl. A61B 34/00; A61B 34/30

(52) U.S. Cl. CPC A61B 34/72; A61B 2034/305

(57) ABSTRACT

Apparatus and methods are described including a robotic unit (20) that includes an end effector (35) and a tool mount (34) configured to securely hold tools (21). Robotic arms (120) rotate the tools through pitch and yaw rotations. An XYZ platform (110) moves the robotic unit (20) along X and Y directions within an XY plane, and along a Z direction that is perpendicular to the XY plane. The XYZ platform (110) includes a first slidable shutter (122) that is configured to cover an interior of the XYZ platform (110) by sliding along the X direction as the robotic unit (20) is moved along the X direction, and a second slidable shutter (123) that is configured to cover an interior of the XYZ platform (110) by sliding along the Y direction as the robotic unit (20) moved along the Y direction. Other applications are also described.

### CROSS-REFERENCES TO RELATED APPLICATIONS

The present application claims priority from U.S. Provisional Patent Application No. 63/285,218 to Korman, filed Dec. 2, 2021, entitled “Robotic unit for microsurgical procedures”, which is incorporated herein by reference.

### FIELD OF EMBODIMENTS OF THE INVENTION

Some applications of the present invention generally relate to medical apparatus and methods. Specifically, some applications of the present invention relate to apparatus and methods for performing microsurgical procedures in a robotic manner.

### BACKGROUND

Cataract surgery involves the removal of the natural lens of the eye that has developed an opacification (known as a cataract), and its replacement with an intraocular lens. Such surgery typically involves a number of standard steps, which are performed sequentially.

In an initial step, the patient's face around the eye is disinfected (typically, with iodine solution), and their face is covered by a sterile drape, such that only the eye is exposed. When the disinfection and draping has been completed, the eye is anesthetized, typically using a local anesthetic, which is administered in the form of liquid eye drops. The eyeball is then exposed, using an eyelid speculum that holds the upper and lower eyelids open. One or more incisions (and typically two or three incisions) are made in the cornea of the eye. The incision(s) are typically made using a specialized blade, which is called a keratome blade. At this stage, lidocaine is typically injected into the anterior chamber of the eye, in order to further anesthetize the eye. Following this step, a viscoelastic injection is applied via the corneal incision(s). The viscoelastic injection is performed in order to stabilize the anterior chamber and to help maintain eye pressure during the remainder of the procedure, and also in order to distend the lens capsule.

In a subsequent stage, known as capsulorhexis, a part of the anterior lens capsule is removed. Various enhanced techniques have been developed for performing capsulorhexis, such as laser-assisted capsulorhexis, zepto-rhexis (which utilizes precision nano-pulse technology), and marker-assisted capsulorhexis (in which the cornea is marked using a predefined marker, in order to indicate the desired size for the capsule opening).

Subsequently, it is common for a fluid wave to be injected via the corneal incision, in order to dissect the cataract's outer cortical layer, in a step known as hydrodissection. In a subsequent step, known as hydrodelineation, the outer softer epi-nucleus of the lens is separated from the inner firmer endo-nucleus by the injection of a fluid wave. In the next step, ultrasonic emulsification of the lens is performed, in a process known as phacoemulsification. The nucleus of the lens is broken initially using a chopper, following which the outer fragments of the lens are broken and removed, typically using an ultrasonic phacoemulsification probe. Further typically, a separate tool is used to perform suction during the phacoemulsification. When the phacoemulsification is complete, the remaining lens cortex (i.e., the outer layer of the lens) material is aspirated from the capsule. During the phacoemulsification and the aspiration, aspirated fluids are typically replaced with irrigation of a balanced salt solution, in order to maintain fluid pressure in the anterior chamber. In some cases, if deemed to be necessary, then the capsule is polished. Subsequently, the intraocular lens (IOL) is inserted into the capsule. The IOL is typically foldable and is inserted in a folded configuration, before unfolding inside the capsule. At this stage, the viscoelastic is removed, typically using the suction device that was previously used to aspirate fluids from the capsule. If necessary, the incision(s) is sealed by elevating the pressure inside the bulbus oculi (i.e., the globe of the eye), causing the internal tissue to be pressed against the external tissue of the incision, such as to force closed the incision.

### SUMMARY

In accordance with some applications of the present invention, a robotic system is configured for use in a microsurgical procedure, such as intraocular surgery. Typically, when used for intraocular surgery, the robotic system includes one or more robotic units (which are configured to hold tools), in addition to an imaging system, one or more displays, and a control-component unit (for example, a control-component unit that includes a pair of control components, such as joysticks), via which one or more operators (e.g., healthcare professionals, such as a physician and/or a nurse) are able to control robotic units. Typically, the robotic system includes one or more computer processors, via which components of the system and the operators operatively interact with each other.

For some applications, a set of tools is provided, each of which includes a universal mount-engagement portion for engaging a tool mount of an end effector of the robotic unit, in accordance with some applications of the present invention. For some applications, the set of tools comprises a universal tool kit for use with the robotic unit that includes all tools that are typically used in a cataract procedure, a different ophthalmic procedure, and/or a different microsurgical procedure. For example, the set of tools typically includes one or more of the following tools: a keratome blade, an eye fixator, a paracentesis knife, a dispersive ophthalmic viscosurgical device (OVD) syringe, a cohesive ophthalmic viscosurgical device (OVD) syringe, a staining syringe (e.g., for staining the anterior lens with a stain such as trypan blue ophthalmic solution), a lidocaine syringe, forceps, a hydrodissection syringe, a phacoemulsification probe, a chopper, an irrigation/aspiration probe, an intraocular lens injector, an antibiotics syringe, and/or a Limbal Relaxing Incision (LRI) knife. For some applications, each of the tools includes one or more markers, which may be used to identify the tools and/or to determine the position and/or orientation of the tool.

Typically, movement of the robotic units (and/or control of other aspects of the robotic system) is at least partially controlled by the one or more operators. For example, the operator may receive images of the patient's eye and the robotic units and/or tools disposed therein, via a display. Based on the received images, the operator typically performs steps of the procedure. For some applications, the operator provides commands to the robotic units via a control-component unit. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, and/or x-y positioning of the imaging system). For some applications, the commands include controlling an intraocular-lens-manipulator tool, for example, such that the tool manipulates the intraocular lens inside the eye for precise positioning of the intraocular lens within the eye.

Typically, the control-component unit includes one or more joysticks that are configured to correspond to respective robotic units of the robotic system. For example, the system may include first and second robotic units, and the control-component unit may include first and second joysticks to be operated by the operators right and left hands. For some applications, the control-component joysticks comprise respective control-component tools therein (in order to replicate the robotic units). Typically, the computer processor determines the XYZ location and orientation of a tip of the control-component tool, and drives the robotic unit such that the tip of the actual tool that is being used to perform the procedure tracks the movements of the tip of the control-component tool.

Typically, the robotic system includes left and right XYZ platforms, each of which has a respective robotic unit disposed thereon, and which are configured to be placed respectively to the left and the right of the portion of the patient's body which is to be operated on, e.g., the patient's eye. Typically, the left and right XYZ platforms are disposed on a stand, and each of the XYZ platforms is rotationally coupled to a rotational axis of the stand. Typically, the rotational axis is disposed along the Z-direction. For some applications, each of the XYZ platforms is configured to rotate (e.g., automatically rotate) about the rotational axis from a first (closed) position to a second (open) position.

Typically, in the closed position the XYZ platforms are disposed relatively close to the patient's eye. For some applications, in this position, the robotic units are disposed close enough to the patient's eye that any further adjustments of the positions of the robotic units relative to the patient's eye are achievable by means of the robotic units moving upon the XYZ platforms, and/or by means of movement of the end effector of the robotic unit, and/or by means of movement of the tool with respect to the end effector. Thus, in the closed position, the XYZ platforms are positioned such that the robotic units are able to perform robotic microsurgery on the patient's eye. Typically, the XYZ platforms are moved away from the patient's eye by being rotated about the rotational axes from the closed position to the open position.

Typically, the XYZ platforms are moved away from the patient's eye for clinical and/or safety-related reasons. For example, in the event that the physician is unable to proceed with the procedure robotically, the rotation of the XYZ platforms provides access to the patient. In this manner, the physician can perform the procedure manually without requiring movement of the patient. Or, for example, if the patient requires immediate attention (e.g., resuscitation), the rotation of the XYZ platforms provides immediate access to the patient. For some applications, the XYZ platforms are moved away from the patient in order to allow sterile drapes to be placed on the robotic system, while avoiding cross-contamination that may be caused by contact with the patient. Similarly, for some applications, the XYZ platforms are moved away from the patient in order to allow the patient to be draped, while avoiding cross-contamination that may be caused by contact with the robotic system. For some applications, the XYZ platforms are moved away from the patient's eye in order to allow the patient to position her/his head within a head support.

For some applications, the robotic unit includes one or more robotic arms that are coupled to an end effector and that are disposed on a robotic-unit base. The robotic arms are typically configured to rotate tools that are coupled to the end effector through pitch and yaw rotations by the one or more robotic arms moving, as described in further detail hereinbelow. For some applications, the robotic-unit base is disposed on the XYZ platform, which is configured to move the robotic unit along X and Y directions within an XY plane, and along a Z direction that is perpendicular to the XY plane.

For some applications, the XYZ platform includes a first slidable shutter that is configured to cover the interior of the XYZ platform by sliding along the X direction as the robotic unit is moved along the X direction, and a second slidable shutter that is configured to cover the interior of the XYZ platform by sliding along the Y direction as the robotic unit is moved along the Y direction. Typically, one of the shutters is disposed upon and perpendicularly with respect to the other shutter. For some applications, the XYZ platform includes a column, which is typically a telescopic column. The robotic unit is typically supported upon the column and the column is configured to extend or retract along the Z direction in order to move the robotic unit along the Z direction.

For some application, movement of the robotic-unit base with respect to the XYZ platform is utilized in order to move the robotic unit closer to the operator. For example, during the performance of a stage of the microsurgical procedure, the position of the robotic-unit base with respect to the XYZ platform may be kept constant with movement of the end effector and the tool being performed via movement of the robotic arms and/or rolling of the tool with respect to the end effector. Between stages of the procedure, the robotic-unit base may be moved closer to the operator by the movement of the robotic-unit base with respect to the XYZ platform, for example, such as to provide access to tool mount to the operator, such that the operator is able to exchange the tool that is in the robotic unit, and/or make a different adjustment to the robotic unit.

The robotic unit typically includes one or more robotic arms that are coupled to the end effector and that are disposed on a robotic-unit base. For some applications, the robotic arms are configured to rotate tools that are coupled to the end effector through pitch and yaw rotations by the one or more robotic arms moving. Typically, the robotic unit is configured to insert tools into the patient's eye such that entry of the tool into the patient's eye is via an incision point, and the tip of the tool is disposed within the patient's eye. Further typically, the robotic unit is configured to move the tip of the tool within the patient's eye in such a manner that entry of the tool into the patient's eye is constrained to remain within the incision point. For some applications, the robotic unit achieves this by being configured such as to define a remote center of motion (“RCM”) of the tool and the RCM of the tool is made to coincide with the incision point. It is noted that for some applications, the robotic unit is configured to move the tip of the tool within the patient's eye in such a manner that entry of the tool into the patient's eye is constrained to remain within an incision zone, rather than an incision point, as described in further detail hereinbelow. For such cases, all aspects of the present application that are described as being applied to an incision point are applied to an incision zone, mutatis mutandis. The term “incision region” is used herein to denote either an incision point or an incision zone.

There is therefore provided, in accordance with some applications of the present invention, apparatus for performing robotic microsurgery on a portion of a body of a patient using one or more tools, the apparatus including:
at least one robotic unit including:
   an end effector;
   a tool mount coupled to the end effector and configured to securely hold the one or more tools; and
   one or more robotic arms coupled to the end effector and which are configured to rotate the one or more tools through pitch and yaw rotations by the one or more robotic arms moving;
at least one XYZ platform configured to move the robotic unit along X and Y directions within an XY plane, and along a Z direction that is perpendicular to the XY plane;
the XYZ platform including:
   a first slidable shutter that is configured to cover an interior of the XYZ platform by sliding along the X direction as the robotic unit is moved along the X direction; and
   a second slidable shutter that is configured to cover an interior of the XYZ platform by sliding along the Y direction as the robotic unit moved along the Y direction.

In some applications, the first slidable shutter is disposed upon and perpendicularly with respect to the second slidable shutter.

In some applications, the XYZ platform includes a column, the robotic unit is supported upon the column, and the column is configured to extend or retract along the Z direction in order to move the robotic unit along the Z direction.

In some applications, each of the slidable shutters is sized such as to provide a range of motion to the robotic unit in each of the X and Y directions of more than 100 mm.

In some applications:
the at least one robotic unit includes two robotic units,
the at least one XYZ platform includes two XYZ platforms, with a respective robotic unit being disposed on each of the XYZ platforms,
a first one of the XYZ platforms and robotic units is configured to be placed on a first side of the portion of the patient's body, and
a second one of the XYZ platforms and robotic units is configured to be placed on a second side of the portion of the patient's body.

In some applications, each of the slidable shutters is configured such that even when the robotic unit is at an extremity of its motion range in either of the X or Y directions, the slidable shutters continue to cover the interior of the XYZ platform.

In some applications, each of the slidable shutters has a length that is greater than a motion range of the robotic unit along a corresponding direction, and has excess shutter length that is configured to roll into an interior of the XYZ platform.

In some applications, the apparatus further includes a rotational axis,
the XYZ platform is rotationally coupled to the rotational axis and is configured to:
automatically rotate about the rotational axis from a first position to a second position such as to provide access to the patient to an operator; and
automatically rotate about the rotational axis from the second position to the first position such as to perform the robotic microsurgery on the portion of the patient's body.

In some applications:
the at least one robotic unit includes two robotic units,
the at least one XYZ platform includes two XYZ platforms, with a respective robotic unit being disposed on each of the XYZ platforms,
a first one of the XYZ platforms and robotic units is configured to be placed on a first side of the portion of the patient's body, and
a second one of the XYZ platforms and robotic units is configured to be placed on a second side of the portion of the patient's body.

In some applications, each of the XYZ platforms is configured to:
automatically rotate about the rotational axis from a first position to a second position such as to provide access to the patient to an operator; and
automatically rotate about the rotational axis from the second position to the first position such as to perform the robotic microsurgery on the portion of the patient's body.

There is further provided, in accordance with some applications of the present invention, apparatus for performing robotic microsurgery on a portion of a body of a patient using one or more tools, the apparatus including:
an end effector;
a tool mount coupled to the end effector and configured to securely hold the one or more tools;
a pair of parallel robotic arms coupled to the end effector and which are configured to rotate the one or more tools through a pitch angular rotation by the pair of parallel robotic arms undergoing a change in pitch; and
a pitch-rotation mechanism including:
   a worm gear coupled to the pair of parallel robotic arms, such that rotation of the worm gear causes the parallel robotic arms to undergo the change in pitch;
   a worm screw that engages the worm gear and is configured to rotate the worm gear; and
   a spring configured to bias the pair of parallel robotic arms in a given direction, such as to reduce backlash of the parallel robotic arms in response to changes in rotational motion of the worm gear.

In some applications, the pitch-rotation mechanism is configured such that, over a full range of pitch rotation of the one or more tools, a relationship between rotations of the worm screw and an angular pitch rotation of the one or more tools is constant.

In some applications, the pitch-rotation mechanism is configured to move the one or more tools through a pitch angular rotation of at least 60 degrees.

In some applications, the parallel arms are configured to constrain movement of the end effector, and thereby constrain the motion of a tool that is within the tool mount, such that as the tool undergoes changes in pitch, a remote center of motion of the tool is maintained.

In some applications, the tool is configured to be inserted into an eye of the patient via an incision point, and wherein the parallel arms are configured to constrain movement of the end effector, and thereby constrain the motion of the tool that is within the tool mount, such that as the tool undergoes changes in pitch, the remote center of motion of the tool is maintained within the incision point.

In some applications, the tool is configured to be inserted into an eye of the patient via an incision zone, and the parallel arms are configured to constrain movement of the end effector, and thereby constrain the motion of the tool that is within the tool mount, such that as the tool undergoes changes in pitch, the remote center of motion of the tool is maintained within the incision zone.

There is further provided, in accordance with some applications of the present invention, apparatus for performing robotic microsurgery on a portion of a body of a patient using one or more tools, the apparatus including:
an end effector;
a tool mount coupled to the end effector and configured to securely hold the one or more tools;
a pair of parallel robotic arms coupled to the end effector and which are configured to rotate the one or more tools through a yaw angular rotation by the pair of parallel robotic arms undergoing a change in yaw; and
a yaw-rotation mechanism including:
   a yaw motor configured to rotate the parallel robotic arms through yaw angular rotation; and
   a first spring that is biased such as to resist rotation of the pair of parallel robotic arms in a first yaw rotational direction from a central yaw rotational position; and
   a second spring that is biased such as to resist rotation of the pair of parallel robotic arms in a second yaw rotational direction from the central yaw rotational position.

In some applications, the first and second springs are configured to reduce backlash during yaw rotational motion of the one or more tools.

In some applications, the yaw-rotation mechanism is configured to move the one or more tools through a pitch angular rotation of plus/minus 25 degrees from the central yaw rotational position.

In some applications, the first and second springs are configured to balance force, such that static load applied to the yaw motor is reduced, thereby reducing deflection of the motor.

In some applications, the first and second springs are configured to counter gravitational pull on the parallel robotic arms, as the parallel robotic arms are rotated in the respective rotational directions from the central yaw rotational position.

In some applications, the first and second springs are configured to stabilize yaw rotational motion of the one or more tools, such that over a full range of the yaw rotational motion of the one or more tools, a relationship between rotations of the yaw motor and an angular yaw rotation of the one or more tools is constant.

In some applications, the yaw-rotation mechanism is configured to constrain movement of the end effector, and thereby constrain the motion of a tool that is within the tool mount, such that as the tool undergoes changes in yaw, a remote center of motion of the tool is maintained.

In some applications, the tool is configured to be inserted into an eye of the patient via an incision point, and the yaw-rotation mechanism is configured to constrain movement of the end effector, and thereby constrain the motion of the tool that is within the tool mount, such that as the tool undergoes changes in yaw, the remote center of motion of the tool is maintained within the incision point.

In some applications, the tool is configured to be inserted into an eye of the patient via an incision zone, and the yaw-rotation mechanism is configured to constrain movement of the end effector, and thereby constrain the motion of the tool that is within the tool mount, such that as the tool undergoes changes in yaw, the remote center of motion of the tool is maintained within the incision zone.

There is further provided, in accordance with some applications of the present invention, apparatus for performing robotic microsurgery on an eye of a patient, the apparatus including: a plurality of tools having different shapes from each other;
an end effector;
a tool mount coupled to the end effector and configured to securely hold the one or more tools;
one or more robotic arms coupled to the end effector and which are configured to move each of the tools while it is held by the tool mount by the one or more robotic arms moving the end effector; and
a computer processor configured to:
   drive the robotic arms to insert the tool into the patient's eye through an incision region;
   receive an input that is indicative of which tool is being held by the tool mount;
   receive a further input indicating that the tool should be retracted from the patient's eye; and
   in response to receiving the further input indicating that the tool should be retracted from the patient's eye, move the tool in such a manner that the tool is removed via the incision region.

In some applications, the computer processor is configured to determine a pattern of movement via which to move the tool such that the tool is removed via the incision region, and to retract the tool in accordance with the determine pattern of movement.

In some applications, the computer processor is further configured to determine a position and/or orientation of the tool with respect to the incision region, and to account for the position and/or orientation of the tool with respect to the incision region such as to remove the tool via the incision region.

There is further provided, in accordance with some applications of the present invention, apparatus for performing robotic microsurgery on a portion of a body of a patient using one or more tools, the apparatus including:
at least one robotic unit including:
   an end effector;
   a tool mount coupled to the end effector and configured to securely hold the one or more tools;
   one or more robotic arms coupled to the end effector and which are configured to rotate the one or more tools through pitch and yaw rotations by the one or more robotic arms moving;
at least one XYZ platform configured to move the one or more robotic arms along X and Y directions along an XY plane, and along a Z direction that is perpendicular to the XY plane;
a rotational axis, the XYZ platform being rotationally coupled to the rotational axis and being configured to:
automatically rotate about the rotational axis from a first position to a second position such as to provide access to the patient to an operator; and
automatically rotate about the rotational axis from the second position to the first position such as to perform the robotic microsurgery on the portion of the patient's body.

In some applications:
the at least one robotic unit includes two robotic units,
the at least one XYZ platform includes two XYZ platforms, with a respective robotic unit being disposed on each of the XYZ platforms,
a first one of the XYZ platforms and robotic units is configured to be placed on a first side of the portion of the patient's body, and
a second one of the XYZ platforms and robotic units is configured to be placed on a second side of the portion of the patient's body.

In some applications, wherein each of the XYZ platforms is configured to:
automatically rotate about the rotational axis from a first position to a second position such as to provide access to the patient to an operator; and
automatically rotate about the rotational axis from the second position to the first position such as to perform the robotic microsurgery on the portion of the patient's body.

There is further provided, in accordance with some applications of the present invention, apparatus for performing robotic microsurgery on an eye of a patient using one or more tools, the apparatus including:
an end effector;
a tool mount coupled to the end effector and configured to securely hold the one or more tools;
one or more robotic arms coupled to the end effector and which are configured to control yaw and pitch angular rotations of the one or more tools, such that a tip of a tool that is held by the tool mount is moved in a desired manner within the patient's eye, while a location of entry of the tool into the patient's eye is maintained within an incision zone;
a control component configured to be moved by an operator such as to move the tool in the desired manner; and
an output unit configured to provide feedback to the operator that is indicative of a location of the location of entry of the tool into the patient's eye within the incision zone.

In some applications, the incision zone is more than 150 percent of a maximum cross section of the tool that passes through the incision zone.

In some applications, the output unit includes a display that shows the incision zone and the location of entry of the tool within the incision zone.

In some applications, the output unit includes an output unit that is configured to generate an alert as the tool is moved in such a manner that the location of the entry of the tool into the patient's eye is close to the edge of the incision zone.

In some applications, the output unit includes a portion of the control component that is configured to provide haptic feedback to the operator.

In some applications, the control component is configured to increase resistance to movement of the control component as the location of the entry of the tool into the patient's eye is closer to the edge of the incision zone.

There is further provided, in accordance with some applications of the present invention, apparatus for performing robotic microsurgery on an eye of a patient, the apparatus including:
a keratome blade;
an end effector;
a tool mount coupled to the end effector and configured to securely hold the one or more keratome blade;
one or more robotic arms coupled to the end effector and which are configured to control movement of the keratome blade by moving the end effector;
a control component configured to be moved by an operator such as to move the keratome blade toward a cornea of the patient's eye;
the control component including an actuation mechanism that is configured to drive the robotic arms to move the keratome blade in a non-linear direction in response to receiving an input from the operator, such as to make an angled incision within the patient's cornea.

The present invention will be more fully understood from the following detailed description of embodiments thereof, taken together with the drawings, in which:

### BRIEF DESCRIPTION OF THE DRAWINGS

FIG. 1 is a schematic illustration of a robotic system that is configured for use in a microsurgical procedure, such as intraocular surgery, in accordance with some applications of the present invention;

FIG. 2 is a schematic illustration of a set of tools each of which includes a universal mount-engagement portion for engaging a tool mount of an end effector of a robotic unit, in accordance with some applications of the present invention;

FIGS. 3A-C are schematic illustrations of respective views of universal mount-engagement portion of tools, in accordance with some applications of the present invention;

FIGS. 4A and 4B are schematic illustrations of respective views of a tool mount in an open state, in accordance with some applications of the present invention;

FIGS. 4C and 4D are schematic illustrations of a tool disposed within the tool mount, and with the tool mount respectively in an open state and in a closed state, in accordance with some applications of the present invention;

FIGS. 5A and 5B are schematic illustrations of oblique views of left and right XYZ platforms of a robotic system in open and closed states respectively, in accordance with some applications of the present invention;

FIGS. 5C and 5D are schematic illustrations of top views of left and right XYZ platforms of a robotic system in open and closed states respectively, in accordance with some applications of the present invention;

FIGS. 6A, 6B, 6C, and 6D are schematic illustrations of a robotic unit of a robotic system at respective X, Y, and Z positions on an XYZ platform, in accordance with some applications of the present invention;

FIG. 7 is a schematic cross-sectional illustration of a robotic unit, in accordance with some applications of the present invention;

FIGS. 8A and 8B are schematic illustrations of respective view of a pitch-rotation mechanism of a robotic unit, in accordance with some applications of the present invention;

FIG. 9 is a schematic illustration of a yaw-rotation mechanism of a robotic unit, in accordance with some applications of the present invention;

FIGS. 10A, 10B, and 10C are schematic illustrations of respective steps of the automatic incision of a patient's cornea with a keratome blade, in accordance with some applications of the present invention;

FIGS. 11A, 11B, and 11C are schematic illustrations of respective steps of the automatic retraction of a tool through an incision point in a patient's cornea, in accordance with some applications of the present invention; and

FIG. 12 is a schematic illustration of a tool inserted through a patient's cornea, such that a tip of the tool is moved in a desired manner within the patient's eye, while a location of entry of the tool into the patient's eye is maintained within an incision zone, in accordance with some applications of the present invention.

### DETAILED DESCRIPTION OF EMBODIMENTS

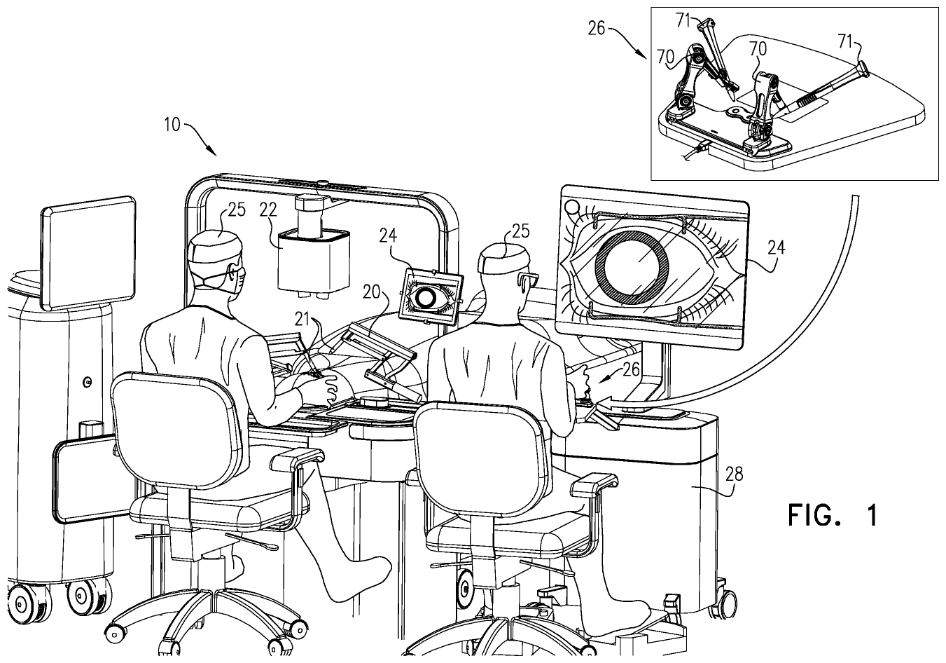

Reference is now made to FIG. 1, which is a schematic illustration of a robotic system 10 that is configured for use in a microsurgical procedure, such as intraocular surgery, in accordance with some applications of the present invention. Typically, when used for intraocular surgery, robotic system 10 includes one or more robotic units 20 (which are configured to hold tools 21), in addition to an imaging system 22, one or more displays 24 and a control-component unit 26 (for example, a control-component unit that includes a pair of control components, such as joysticks 70, as shown in the enlarged portion of FIG. 1), via which one or more operators 25 (e.g., healthcare professionals, such as a physician and/or a nurse) is able to control robotic units 20. Typically, robotic system 10 includes one or more computer processors 28, via which components of the system and operator(s) 25 operatively interact with each other. The scope of the present application includes mounting one or more robotic units in any of a variety of different positions with respect to each other.

Typically, movement of the robotic units (and/or control of other aspects of the robotic system) is at least partially controlled by one or more operators 25 (e.g., healthcare professionals, such as a physician and/or a nurse). For example, the operator may receive images of the patient's eye and the robotic units, and/or tools disposed therein, via display 24. Typically, such images are acquired by imaging system 22. For some applications, imaging system 22 is a stereoscopic imaging device and display 24 is a stereoscopic display. Based on the received images, the operator typically performs steps of the procedure. For some applications, the operator provides commands to the robotic units via control-component unit 26. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, and/or x-y positioning of the imaging system). For some applications, the commands include controlling an intraocular-lens-manipulator tool, for example, such that the tool manipulates the intraocular lens inside the eye for precise positioning of the intraocular lens within the eye.

Typically, the control-component unit includes one or more control components (e.g., joysticks 70) that are configured to correspond to respective robotic units 20 of the robotic system. For example, as shown, the system may include first and second robotic units, and the control component unit may include first and second joysticks, as shown. For some applications, the control-component joysticks comprise respective control-component tools 71 therein (in order to replicate the robotic units), as shown in FIG. 1. Typically, the computer processor determines the XYZ location and orientation of the tip of the control-component tool 71, and drives the robotic unit such that the tip of the actual tool 21 that is being used to perform the procedure tracks the movements of the tip of the control-component tool.

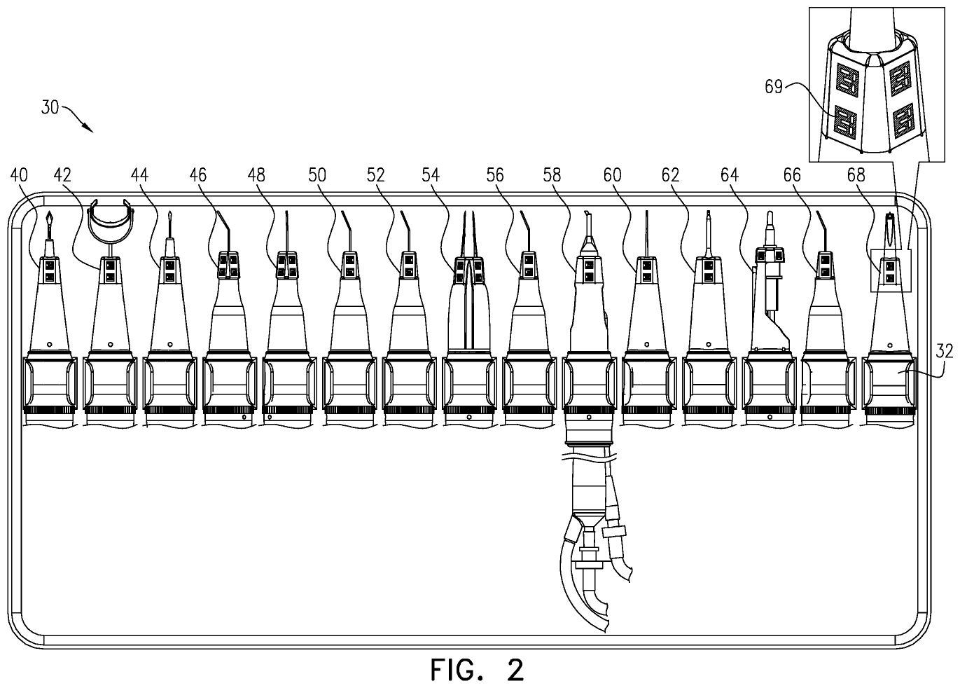

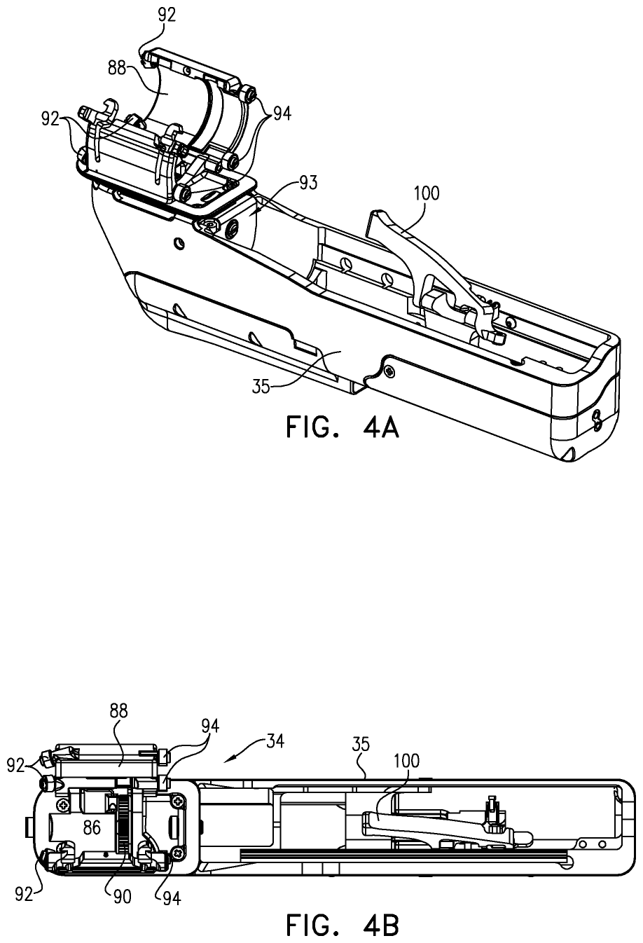

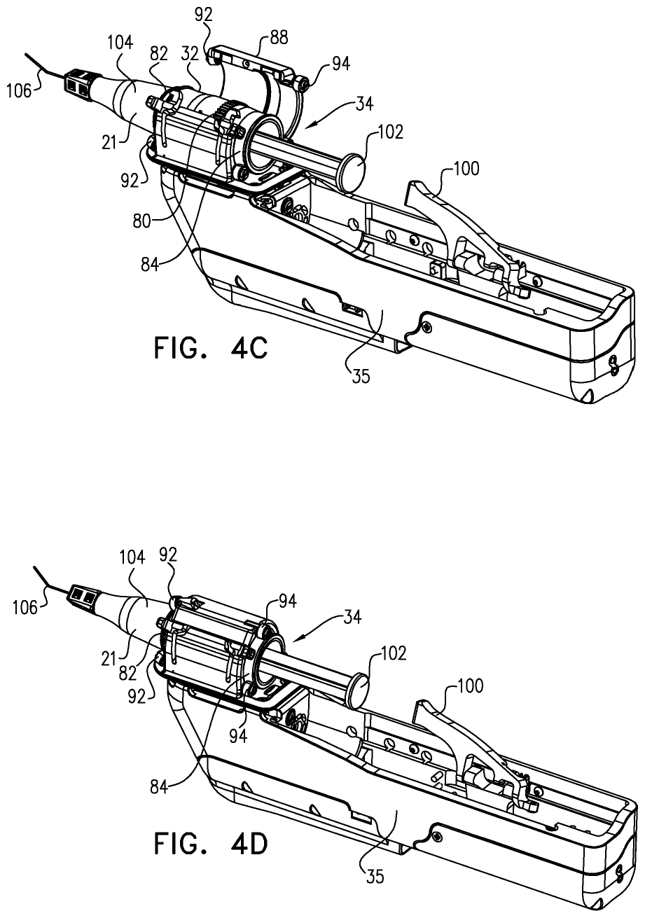

Reference is now made to FIG. 2 is a schematic illustration of a set 30 of tools each of which includes a universal mount-engagement portion 32 for engaging a tool mount 34 of an end effector 35 (tool mount and end effector being shown in FIGS. 4A-D) of robotic unit 20, in accordance with some applications of the present invention. For some applications, set 30 of tools 21 comprises a universal tool kit for use with the robotic unit 20 that includes all tools that are typically used in a cataract procedure, a different ophthalmic procedure, and/or a different microsurgical procedure. For example, as shown in FIG. 2, the set of tools typically includes one or more of the following tools: a keratome blade 40, an eye fixator 42, a paracentesis knife 44, a dispersive ophthalmic viscosurgical device (OVD) syringe 46, a cohesive ophthalmic viscosurgical device (OVD) syringe 48, a staining syringe 50 (e.g., for staining the anterior lens with a stain such as trypan blue ophthalmic solution), a lidocaine syringe 52, forceps 54, a hydrodissection syringe 56, a phacoemulsification probe 58, a chopper 60, an irrigation/aspiration probe 62, an intraocular lens injector 64, an antibiotics syringe 66, and/or a Limbal Relaxing Incision (LRI) knife 68. For some applications, each of the tools includes one or more markers 69, which may be used to identify the tools and/or to determine the position and/or orientation of the tool. Some functions of markers 69 are described in further detail hereinbelow.

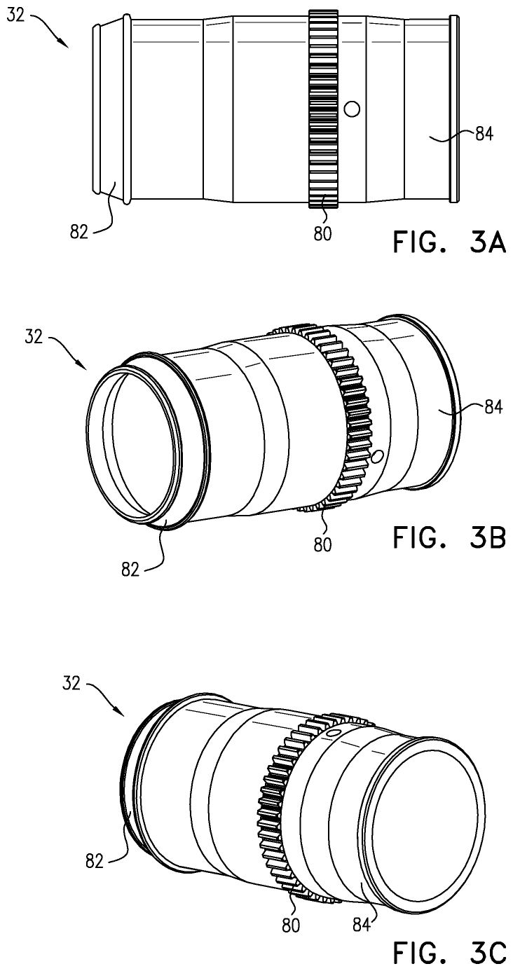

Reference is now made to FIGS. 3A-C, which are schematic illustrations of respective views of universal mount-engagement portion 32 of tools 21, in accordance with some applications of the present invention. Reference is also made to FIGS. 4A and 4B, which are schematic illustrations of respective views of tool mount 34 of end effector 35 in an open state, as well as to FIGS. 4C and 4D, which are schematic illustrations of a tool 21 disposed within the tool mount, with the tool mount respectively in an open state and in a closed state, in accordance with some applications of the present invention. Tool mount 34 is typically coupled to, or formed integrally with, an end effector 35 of the robotic unit.

Typically, mount-engagement portion 32 of tools 21 comprises a sleeve that is disposed around the outside of each of the tools. The sleeve includes a gear wheel 80, as well as a front recess 82 and a rear recess 84. For some applications, at least one of the front and rear recesses has a frustoconical shape. For example, as shown, front recess 82 has a frustoconical shape. Typically, tool mount 34 includes a socket 86 for receiving the tool, as well as a tool-securement cover 88 that is hingedly coupled to the tool-receiving socket and that is configured to secure the tool within the tool-receiving socket. Typically, in order to place the tool in the tool mount, the tool-securement cover is opened (as shown in FIGS. 4A-B). The tool is then placed within socket 86 (as shown in FIG. 4C), before the tool-securement cover 88 is closed such as to secure the tool within the tool-receiving socket (as shown in FIG. 4D).

Typically, mount-engagement portion 32 is sized such that when the tool is secured within the tool-receiving socket (as described in further detail hereinbelow), gear wheel 80 of the mount-engagement portion is positioned such as to engage a gear wheel 90 of the tool mount. A motor 93 of robotic unit 20 is typically configured to drive the tool to roll with respect to the tool mount by driving gear wheel 90 to roll gear wheel 80, to thereby cause the tool to roll. For some applications, the tool is secured within the tool-receiving socket by front rollers 92 being placed within front recess 82 and rear rollers 94 being placed within rear recess 84. (It is noted that in order to place the rollers into the recesses, the tool may be moved with respect to the rollers as an alternative to, or in addition to, the rollers being moved with respect to the tool.) As noted above, for some applications, at least one of the front and rear recesses has a frustoconical shape. Typically, for such applications, the rollers that are configured to be placed within the frustoconical recess are disposed at an angle with respect to the axis of the tool. For example, as shown, front recess 82 has a frustoconical shape, and front rollers 92 are disposed at an angle with respect to the axis of the tool, such as to conform to the shape of the frustoconical recess.

As noted above, robotic unit 20 is typically configured to drive the tool to roll with respect to the tool mount by driving gear wheel 90 to roll gear wheel 80, to thereby cause the tool to roll. Typically, the insertion of the front rollers 92 into front recess 82 and rear rollers 94 into rear recess 84 is such that the rollers act as radial bearings during rolling of the tool. Further typically, the insertion of the front rollers 92 into front recess 82 and rear rollers 94 into rear recess 84 is such as to allow the tool to roll with respect to the tool mount while securely holding the tool in place with respect to the tool mount both radially and axially.

For some applications, tool 21 is configured to be actuated to perform a function via a linear tool-actuation arm 100, which is disposed on end effector 35 and is configured to push a portion of the tool axially. Typically, the linear tool-actuation arm pushes the portion of the tool distally with respect to the tool mount and the mount-engagement portion (i.e., such that the portion of the tool moves distally relative to the tool mount and the mount-engagement portion. For example, as shown in FIG. 4C-D, the tool that is placed within the tool mount is a syringe (e.g., dispersive ophthalmic viscosurgical device (OVD) syringe 46, cohesive ophthalmic viscosurgical device (OVD) syringe 48, staining syringe 50, lidocaine syringe 52, hydrodissection syringe 56, intraocular lens injector 64, and/or antibiotics syringe 66). Typically, the syringe includes a plunger 102, a barrel 104, and a cannula 106. In such cases, the linear tool-actuation arm is configured to push plunger 102 of the syringe axially in a forward direction. For some applications, a portion of the tool is configured to be moved with respect to the patient's eye by linear tool-actuation arm 100 pushing a portion of the tool axially. As described hereinabove, for some applications, front recess 82 has a frustoconical shape, and front rollers 92 are disposed at an angle with respect to the axis of the tool. Typically, by being configured in this manner, the front rollers are configured to counter forces that would otherwise push the entire tool axially forward relative to the tool mount. For example, when the linear tool-actuation arm pushes the plunger of the syringe axially in the forward direction, the front roller typically applies a counterforce to the syringe to prevent barrel 104 of the syringe from being pushed axially forward relative to the tool mount. More generally, a recess having a frustoconical shape typically provides additional axial stability to the tool relative to if the recess were to be coaxial with the axis of the tool.

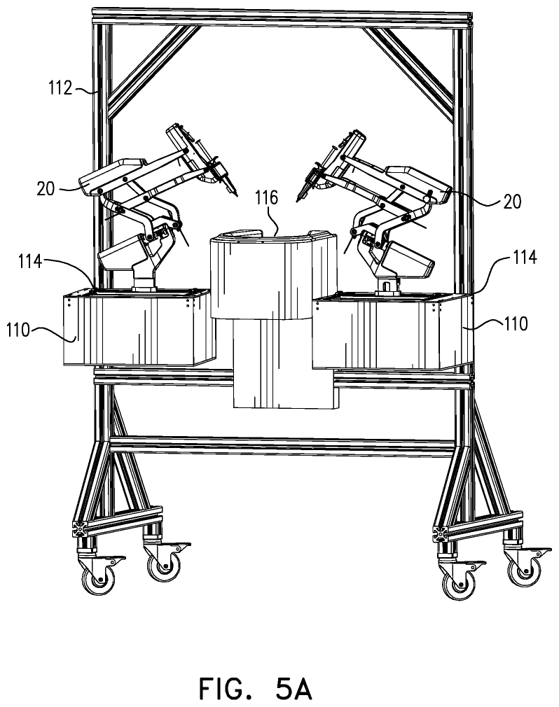

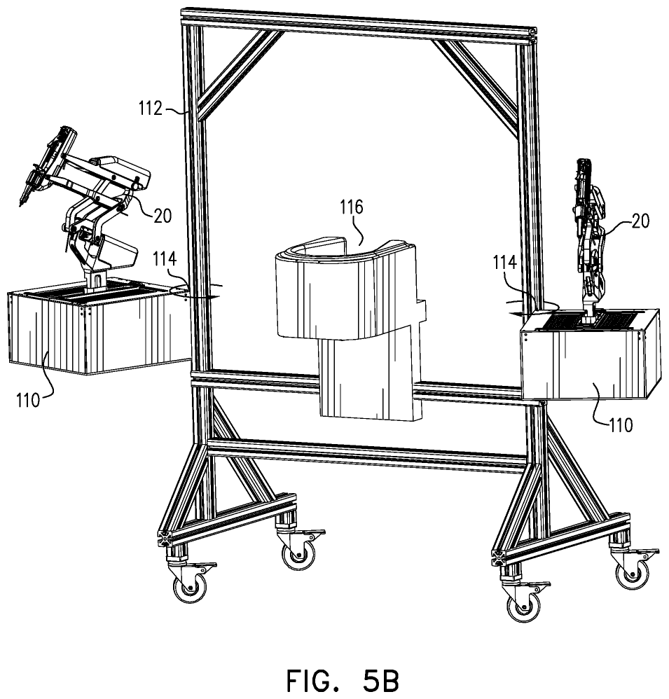

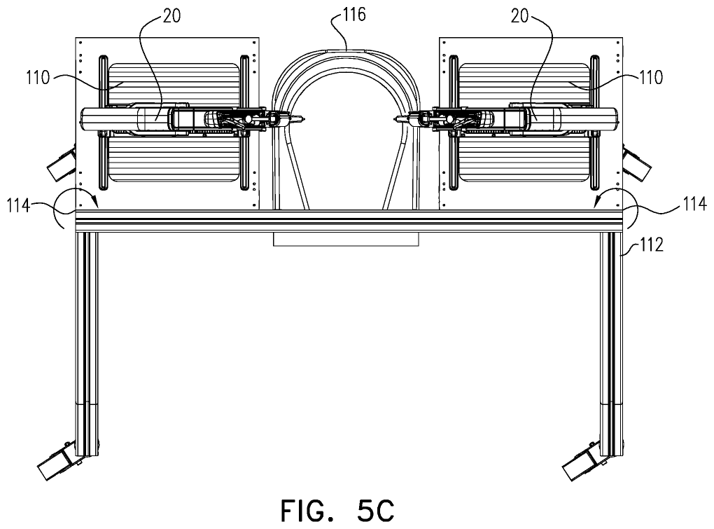

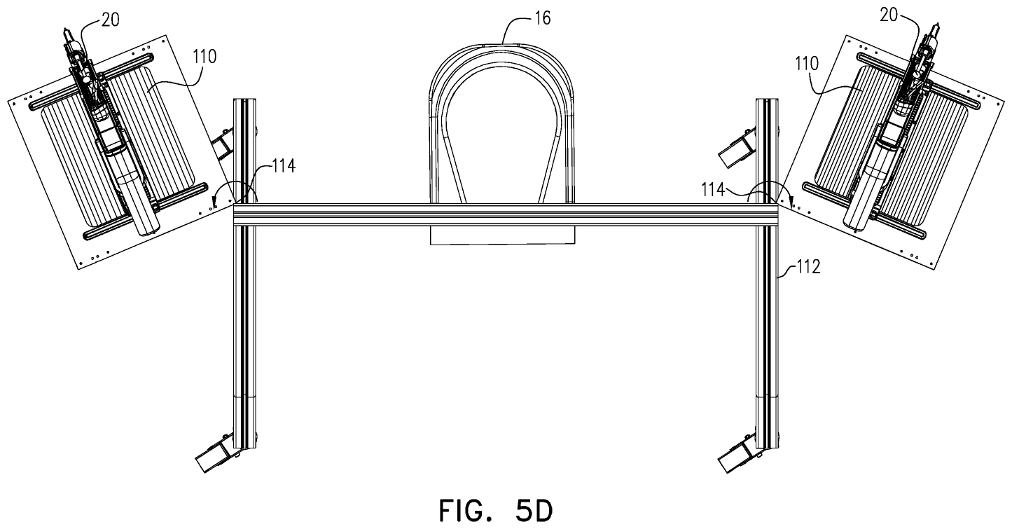

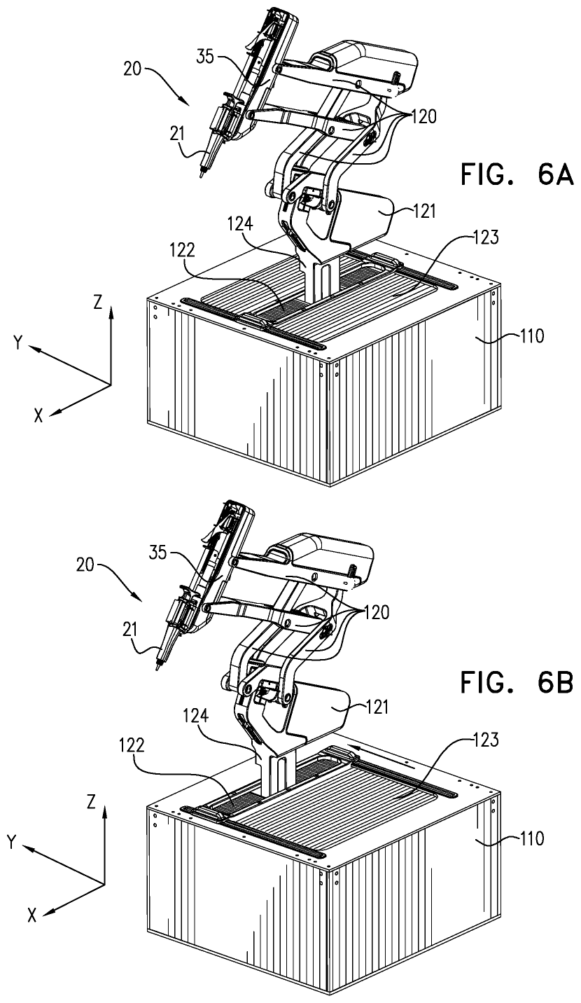

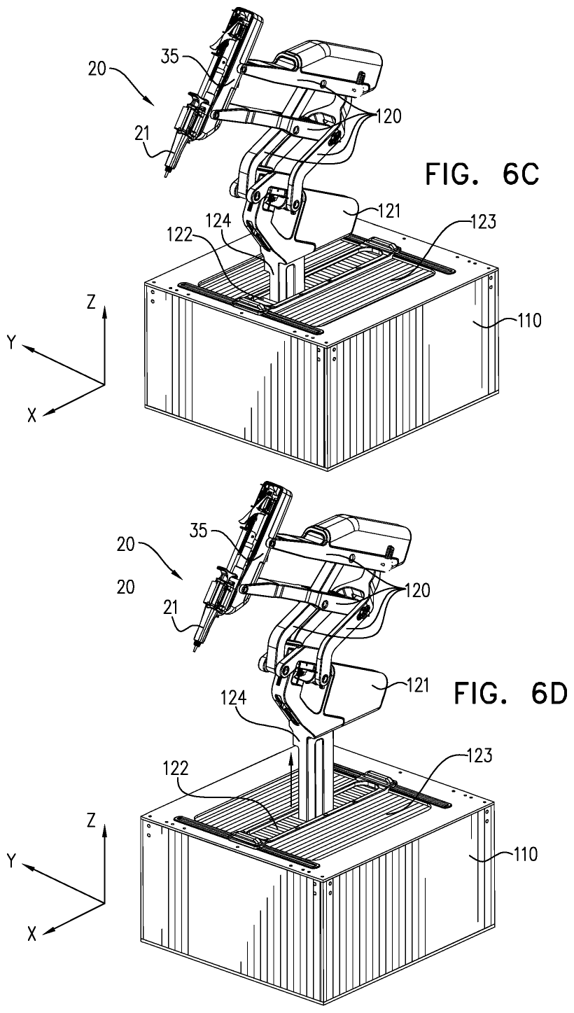

Reference is now made to FIGS. 5A and 5B, which are schematic illustrations of oblique views of left and right XYZ platforms 110 of robotic system 10, in open and closed states respectively, in accordance with some applications of the present invention. Reference is also made to FIGS. 5C and 5D, which are schematic illustrations of top views of left and right XYZ platforms 110 of robotic system 10 in open and closed states respectively, in accordance with some applications of the present invention. Reference is additionally made to FIGS. 6A, 6B, 6C, and 6D, which are schematic illustrations of robotic unit 20 of robotic system 10 at respective X, Y, and Z positions on XYZ platform 110, in accordance with some applications of the present invention.

Typically, robotic system 10 includes left and right XYZ platforms, each of which has a respective robotic unit disposed thereon, and which are configured to placed respectively to the left and the right of the portion of the patient's body which is to be operated on, e.g., the patient's eye. Typically, the left and right XYZ platforms are disposed on a stand 112, and each of the XYZ platforms is rotationally coupled to a rotational axis 114 of the stand. Typically, the rotational axis is disposed along the Z-direction. As shown in the transition from FIG. 5A to FIG. 5B, as well as in the transition from FIG. 5C to FIG. 5D, for some applications, each of the XYZ platforms is configured to rotate (e.g., automatically rotate) about the rotational axis from a first (closed) position to a second (open) position.

Typically, in the closed position the XYZ platforms are disposed relatively close to the patient's eye. For some applications, in this position, the robotic units are disposed close enough to the patient's eye that any further adjustments of the positions of the robotic units relative to the patient's eye are achievable by means of the robotic units moving upon the XYZ platforms (as shown in FIGS. 6A-D), and/or by means of movement of the end effector of the robotic unit, and/or by means of movement of the tool with respect to the end effector. Thus, in the closed position, the XYZ platforms are positioned such that the robotic units are able to perform robotic microsurgery on the patient's eye. Typically, the XYZ platforms are moved away from the patient's eye by being rotated about rotational axes 114 from the closed position to the open position. For some applications, the rotation of the XYZ platforms about rotational axes 114 is performed automatically, e.g., in response to an input by operator 25 to computer processor 28. Typically, whether the XYZ platforms are rotated automatically or manually, the robotic system will only allow the XYZ platforms to rotate in one or both of the directions of rotation when a set of operating parameters are satisfied, for safety reasons.

Typically, the XYZ platforms are moved away from the patient's eye for clinical or safety-related reasons, and to provide access to the patient by the operator. For example, in the event that the physician is unable to proceed with the procedure robotically, the rotation of the XYZ platforms provides access to the patient. In this manner, the physician can perform the procedure manually without requiring movement of the patient. Or, for example, if the patient requires immediate attention (e.g., resuscitation), the rotation of the XYZ platforms provides immediate access to the patient. For some applications, the XYZ platform is moved away from the patient in order to allow sterile drapes to be placed on the robotic system, while avoiding cross-contamination that may be caused by contact with the patient. Similarly, the XYZ platform is moved away from the patient in order to allow the patient to be draped, while avoiding cross-contamination that may be caused by contact with the robotic system. For some applications, the XYZ platforms are moved away from the patient's eye in order to allow the patient to position her/his head within a head support 116.

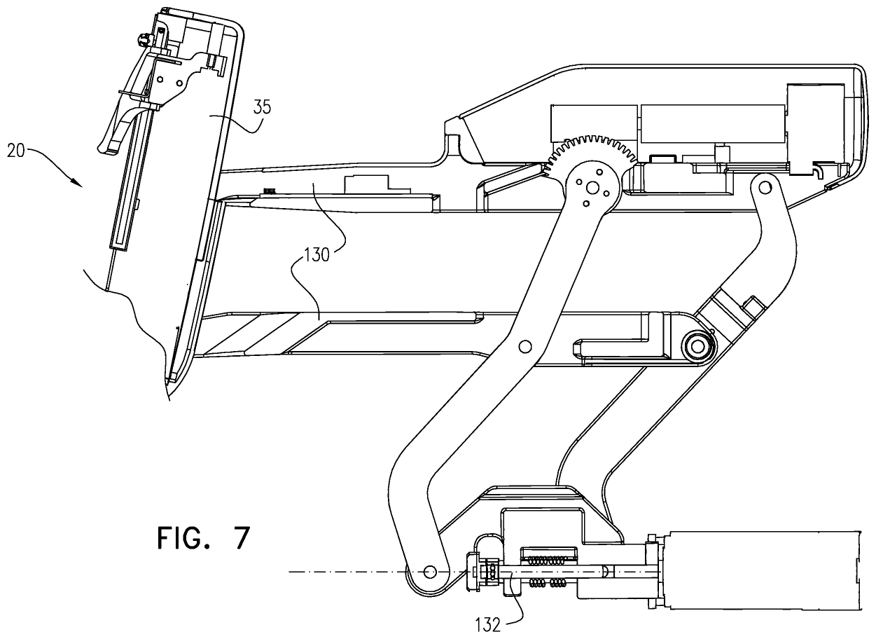

Referring to FIG. 6A-D, for some applications, robotic unit 20 includes one or more robotic arms 120 that are coupled to end effector 35 and that are disposed on a robotic-unit base 121. The robotic arms are typically configured to rotate tools that are coupled to the end effector through pitch and yaw rotations by the one or more robotic arms moving, as described in further detail hereinbelow with reference to FIGS. 7-9. For some applications, the robotic-unit base is disposed on XYZ platform 110, which is configured to move the robotic unit along X and Y directions within an XY plane, and along a Z direction that is perpendicular to the XY plane. For example, the transition from FIG. 6A to 6B shows the robotic unit being moved along the Y-axis, the transition from FIG. 6A to 6C shows the robotic unit being moved along the X-axis, and the transition from FIG. 6A to 6D shows the robotic unit being moved along the Z-axis.

For some applications, XYZ platform 110 includes a first slidable shutter 122 that is configured to cover the interior of the XYZ platform by sliding along the X direction as the robotic unit is moved along the X direction, and a second slidable shutter 123 that is configured to cover the interior of the XYZ platform by sliding along the Y direction as the robotic unit is moved along the Y direction. Typically, one of the shutters is disposed upon and perpendicularly with respect to the other shutter. For example, as shown, first slidable shutter 122 (which is configured to cover the interior of the XYZ platform by sliding along the X direction as the robotic unit is moved along the X direction) is disposed upon and perpendicularly with respect to second slidable shutter 123 (which is configured to cover the interior of the XYZ platform by sliding along the Y direction as the robotic unit is moved along the Y direction).

Typically, each of the shutters is sized such as to provide a range of motion to the robotic unit in each of the X and Y directions of more than 100 mm, e.g., more than 125 mm, more than 140 mm, or more than 150 mm. Further typically, the shutters are configured such that even when the robotic unit is at an extremity of its motion range in either of the X or Y directions, the shutters continue to cover the interior of the XYZ platform. Typically, this is achieved by the shutters being sized such that they are longer than the above-described motion range (for example, the shutters may be more than 180 mm, or more than 200 mm in length), and by the excess shutter length being configured to roll into the interior of the XYZ platform. In this manner, when the robotic unit is at an extremity of it motion range in either of the X or Y directions, the excess length of the corresponding shutter covers the interior of the XYZ platform at the opposite end of the XYZ platform from the location of the robotic unit.

For some applications, the XYZ platform 110 includes a column 124, which is typically a telescopic column. The robotic unit is typically supported upon the column and the column is configured to extend or retract along the Z direction in order to move the robotic unit along the Z direction, as shown in the transition from FIG. 6A to 6D.

For some applications, movement of the robotic-unit base with respect to the XYZ platform is utilized in order to move the robotic unit closer to the operator. For example, during the performance of a stage of the microsurgical procedure, the position of the robotic-unit base with respect to the XYZ platform may be kept constant with movement of the end effector and the tool being performed via movement of the robotic arms (e.g., as described hereinbelow with reference to FIGS. 7-9) and/or rolling of the tool with respect to the end effector (e.g., as described hereinabove with reference to FIGS. 4A-D). Between stages of the procedure, the robotic-unit base may be moved closer to the operator by the movement of the robotic-unit base with respect to the XYZ platform, for example, such as to provide access to tool mount 34 to the operator, such that the operator is able to exchange the tool that is in the robotic unit, and/or make a different adjustment to the robotic unit.

Reference is now made to FIG. 7, which is a schematic cross-sectional illustration of robotic unit 20, in accordance with some applications of the present invention. As described hereinabove, robotic unit 20 typically includes one or more robotic arms 120 that are coupled to end effector 35 and that are disposed on a robotic-unit base 121. For some applications, the robotic arms are configured to rotate tools that are coupled to the end effector through pitch and yaw rotations by the one or more robotic arms moving.

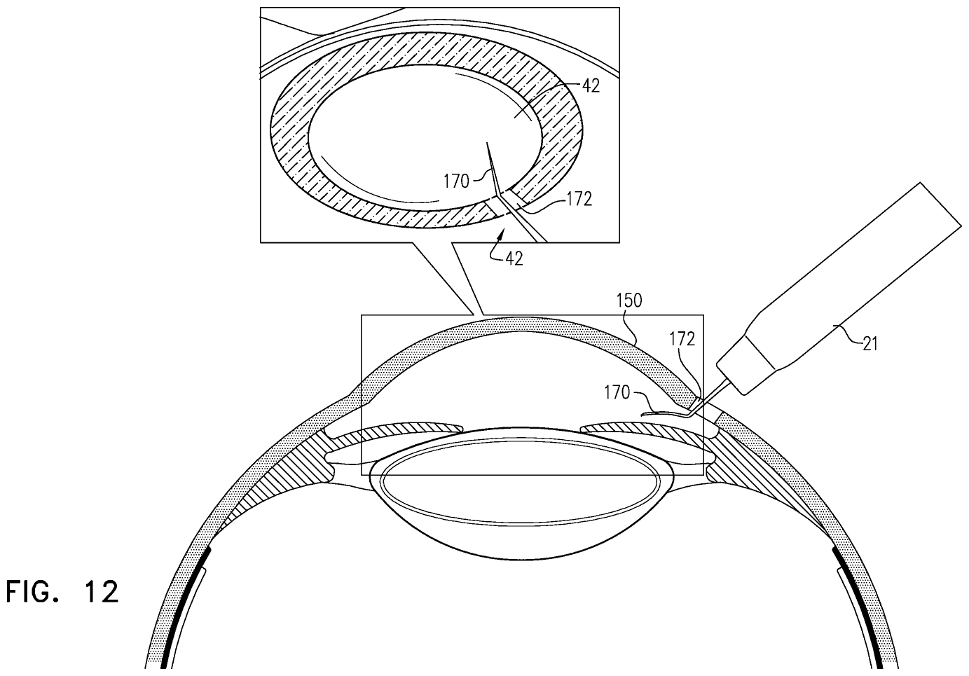

Typically, the robotic unit is configured to insert tools 21 into the patient's eye such that entry of the tool into the patient's eye is via an incision point, and the tip of the tool is disposed within the patient's eye. Further typically, the robotic unit is configured to move the tip of the tool within the patient's eye in such a manner that entry of the tool into the patient's eye is constrained to remain within the incision point. For some applications, the robotic unit achieves this by being configured such as to define a remote center of motion (“RCM”) of the tool and the RCM of the tool is made to coincide with the incision point. It is noted that for some applications, the robotic unit is configured to move the tip of the tool within the patient's eye in such a manner that entry of the tool into the patient's eye is constrained to remain within an incision zone, rather than an incision point, as described in further detail hereinbelow, with reference to FIG. 12. For such cases, all aspects of the present application that are described as being applied to an incision point are applied to an incision zone, mutatis mutandis. The term “incision region” is used herein to denote either an incision point or an incision zone.

Typically, the robotic arms have a parallelogram structure with one or more pairs of parallel arms (e.g., two pairs of parallel arms as shown). Typically, the parallelogram structure includes a pair of parallel arms 130 that are disposed one above the other along the Z direction (at least when the robotic arms are disposed in a central orientation with respect to yaw rotation). Further typically, the parallel arms function to constrain movement of the end effector, and thereby constrain the motion of tool 21, such that as the tool undergoes changes in pitch, the RCM of the tool is maintained. For some applications, the robotic arms are configured to rotate tools that are coupled to the end effector through yaw rotation by rotating about an axis 132. Typically, this results in the tool rotating about a virtual axis (which is the extrapolation of axis 132). Further typically, the rotation of the tool about the virtual axis is such that as the tool undergoes changes in yaw angular position, the RCM of the tool is maintained.

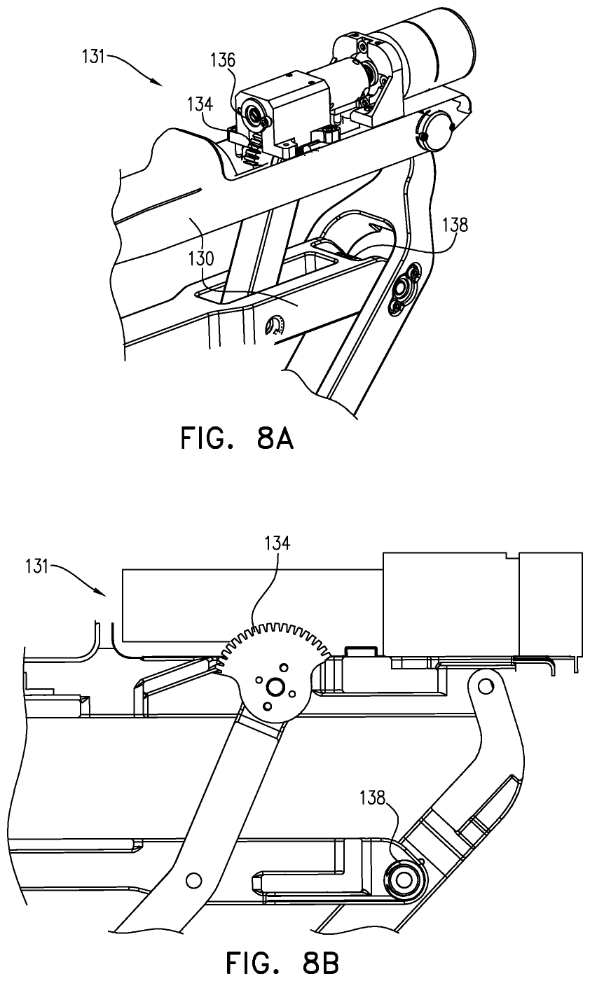

Reference is now made to FIGS. 8A and 8B, which are schematic illustrations of respective views of a pitch-rotation mechanism 131 of robotic unit 20, in accordance with some applications of the present invention. FIG. 8A shows an oblique view, while FIG. 8B shows a cross-sectional view. For some applications, a worm gear 134 is coupled to the pair of parallel robotic arms 130, such that rotation of the worm gear causes the parallel robotic arms to undergo the change in pitch. Typically, a worm screw 136 engages the worm gear and is configured to rotate the worm gear. Further typically, a spring 138 is configured to bias the pair of parallel robotic arms in a given direction, such as to reduce backlash of the parallel robotic arms in response to changes in rotational motion of the gear wheel. By configuring the pitch-rotation mechanism in the above-described manner, it is typically the case that over the full range of the pitch rotation, the relationship between rotations of the worm screw and the angular pitch rotation of the tool is constant. This is in contrast to other pitch-rotation mechanisms that rely, for example, upon a linear driving mechanism, in which the relationship between movement of the linear driving mechanism and the angular pitch rotation of the tool varies over the range of the pitch rotation.

For some applications, the robotic unit is configured to move the tool mount (and thereby move the tool) through a pitch angular rotation of at least 60 degrees. Typically, movement of the tool mount through the pitch angular rotation is performed starting from a starting pitch of approximately 20 degrees (e.g., between 15 and 25 degrees) from the x-y plane.

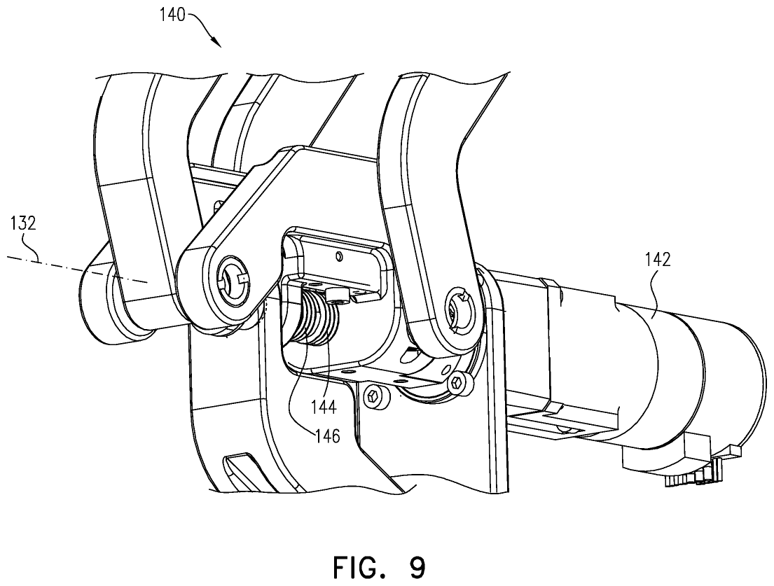

Reference is now made to FIG. 9, which is a schematic illustration of a yaw-rotation mechanism 140 of robotic unit 20, in accordance with some applications of the present invention. Typically, the robotic unit defines a central yaw position in which parallel arms 130 are disposed one above the other. As described hereinabove, for some applications, the robotic arms are configured to rotate tools that are coupled to the end effector through yaw rotation by rotating about axis 132. Typically, this results in the tool rotating about a virtual axis (which is the extrapolation of axis 132). Typically, the yaw-rotation mechanism includes a yaw motor 142 that is configured to rotate the parallel robotic arms through yaw angular rotation. For some applications, the yaw-rotation mechanism additionally includes a first spring 144 that is biased such as to resist rotation of the pair of parallel robotic arms in a first yaw rotational direction (e.g., in the clockwise direction about axis 132) from a central yaw rotational position, and a second spring 146 that is biased such as to resist rotation of the pair of parallel robotic arms in a second yaw rotational direction (e.g., in the counterclockwise direction about axis 132) from the central yaw rotational position.

Typically, as the parallel robotic arms are rotated from the central yaw rotational position, gravity acts to pull the arms further from the central yaw rotational position. The first and second springs are typically configured to counter this gravitational pull, as the arms are rotated in the respective directions. This typically stabilizes the yaw rotational motion, such that over the full range of the yaw rotation, the relationship between rotations of the yaw motor and the yaw rotational motion of the tool is constant. Further typically, the first and second springs act to reduce backlash during yaw rotational motion. For some applications, the first and second springs balance force, such that the static load applied to the motor is reduced, thereby reducing its deflection (which is due to its finite stiffness). Typically, this results in the yaw rotational motion of the tool being smoother than in the absence of the springs.

For some applications, the robotic unit is configured to move the tool mount (and thereby move the tool) through a yaw angular rotation of plus/minus 25 degrees from the central yaw rotational position.

To summarize the descriptions of FIGS. 1-9, typically robotic system 10 is configured to provide several different types of motion, any combination of which may be implemented in accordance with some applications of the present invention. The types of motion provided by the robotic system may include any one of the following or any combination thereof:
Movement of the XYZ platforms between the open and closed positions. Typically, the XYZ platforms are moved away from the patient's eye for clinical or safety-related reasons, and to provide access to the patient by the operator. For example, in the event that the physician is unable to proceed with the procedure robotically, the rotation of the XYZ platforms provides access to the patient. In this manner, the physician can perform the procedure manually without requiring movement of the patient. Or, for example, if the patient requires immediate attention (e.g., resuscitation), the rotation of the XYZ platforms provides immediate access to the patient. For some applications, the XYZ platform is moved away from the patient in order to allow sterile drapes to be placed on the robotic system, while avoiding cross-contamination that may be caused by contact with the patient. Similarly, the XYZ platform is moved away from the patient in order to allow the patient to be draped, while avoiding cross-contamination that may be caused by contact with the robotic system. For some applications, the XYZ platforms are moved away from the patient's eye in order to allow the patient to position her/his head within a head support 116.
Movement of the robotic-unit base in the X, Y, and Z directions with respect to XYZ platform. This is typically implemented between stages of the procedure in order to provide access to the robotic unit to an operator. For example, this might be implemented in order to move the robotic unit closer to a nurse (e.g., to facilitate tool changing).
Pitch and yaw angular rotations of the end effector (and thereby pitch and yaw angular rotations of the tool). Typically, such movements are performed during the procedure, in order to insert tools into the patient's eye, and/or in order to move the tip of the tool within the patient's eye. Further typically, these movements are performed such that while the tip of the tool is moved within the patient's eye, entry of the tool into the patient's eye is constrained to remain within an incision region (e.g., an incision point or an incision zone). For some applications, the robotic unit achieves this by being configured such as to define a remote center of motion (“RCM”) of the tool and the RCM of the tool is made to coincide with the incision region.
Rolling of the tool with respect to the end effector.
Linear motion of a portion of the tool by the portion of the tool being pushed axially by linear tool-actuation arm 100. For example, this movement may be performed in order to actuate the tool to perform a function. Alternatively or additionally, this action may be performed in order to move a portion of the tool (e.g. the tip of the tool) with respect to the patient's eye.

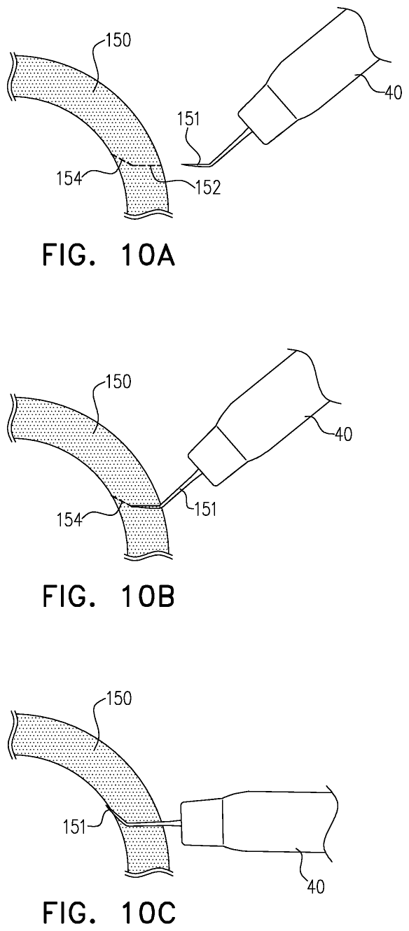

Reference is now made to FIGS. 10A, 10B, and 10C, which are schematic illustrations of respective steps of the automatic incision of a patient's cornea 150 with a keratome blade 40, in accordance with some applications of the present invention. As described hereinabove, for some applications, the operator provides commands to the robotic units via control-component unit 26 (shown in FIG. 1). Typically, such commands include commands that control the position and/or orientation of tools that are disposed within the robotic units, and/or commands that control actions that are performed by the tools. For some applications, control component (e.g., joystick 70 and/or control-component tool 71) is configured to be moved by an operator such as to move the keratome blade toward cornea 150. Typically, the control component includes an actuation mechanism (e.g., a button or a pressure-sensitive pad). For some applications, in response to the operator actuating the actuation mechanism when the keratome blade is disposed within the tool mount, the computer processor is configured to drive the robotic arms to move the keratome blade in a non-linear direction, such as to make an angled incision within the patient's cornea. As shown in the transition from FIG. 10A to 10B, a tip 151 of the keratome blade is typically inserted at a first angle in order to cut a first portion 152 of the patient's cornea. As shown in the transition from FIG. 10B to 10C, the angle of the tip of the keratome blade is the adjusted to a second angle to cut a second portion 154 of the patient's cornea, such that the second portion of the incision is disposed at an angle to the first portion of the incision. Typically, this results in an incision that conforms with the natural curvature of the patient's cornea.

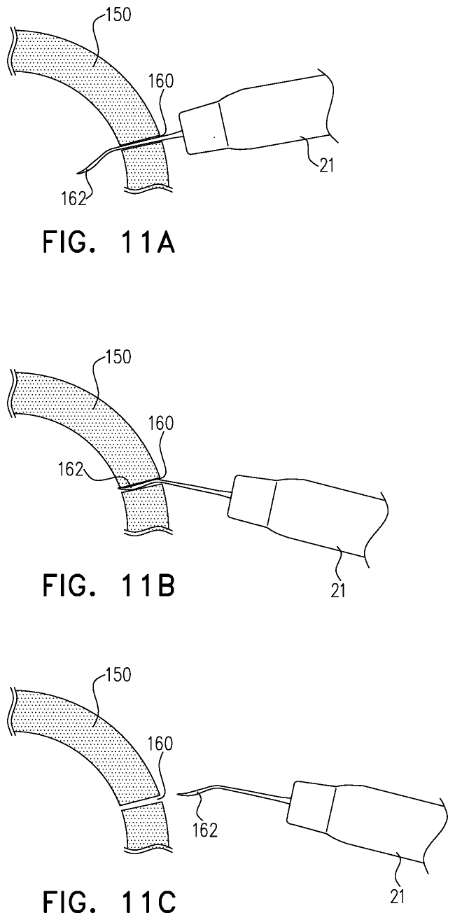

Reference is now made to Reference FIGS. 11A, 11B, and 11C, which are schematic illustrations of respective steps of the automatic retraction of a tool through an incision point in a patient's cornea, in accordance with some applications of the present invention. As described hereinabove with reference to FIG. 2 typically robotic unit 20 is configured to be used with a plurality of tools 21 that have different shapes from each other. Typically, the robotic unit is configured to insert tools 21 into the patient's eye such that entry of the tool into the patient's eye is via an incision region 160 (e.g., an incision point or an incision zone), and the tip 162 of the tool is disposed within the patient's eye. Further typically, the robotic unit is configured to move the tip of the tool within the patient's eye in such a manner that entry of the tool into the patient's eye is constrained to remain within the incision region. Typically, it is desirable to retract the tools from the patient's eye via the incision region. However, in some cases, the portion of the tool that is in the patient's eye has a non-linear shape. Moreover, since the tools have different shapes and sizes from each other, in order to remove any given tool from the patient's eye via the incision region requires a particular pattern of movement, with the required pattern of movement also being dependent on the position and orientation of the tool with respect to the incision region.

In view of the above, in accordance with some applications of the present invention, computer processor 28 receives an input that is indicative of a tool that is coupled to the end effector. For example, the operator may input an indication of the tool into the computer processor. Alternatively or additionally, each of the tools may have a tool-identification component (e.g., marker 69 (shown in FIG. 2)), and the computer processor may be configured to automatically derive which tool is currently coupled to the end effector by identifying the tool-identification component within an image of the tool. Further alternatively or additionally, the computer processor may be configured to automatically derive which tool is currently coupled to the end effector by analyzing an image of the tool, even without using the tool-identification component. In response to receiving an input indicating that the tool should be retracted from the patient's eye, the computer processor drives the robotic unit to move the tool in such a manner that the tool is removed via the incision region through which the tool was inserted. One example of this is shown in FIGS. 11A-C, which schematically illustrate the removal of a portion of tool 21 (in this example shown, tip 162) having a non-linear shape being removed through an incision region 160.

For some applications, the computer processor additionally determines the position and/or orientation of the tool with respect to the incision point, and accounts for the position and/or orientation of the tool with respect to the incision region in driving the robotic unit to remove the tool via the incision region. For example, the computer processor may be configured to automatically derive the position and/or orientation of the tool with respect to the incision region by identifying the tool-identification component (e.g., marker 69) and the incision region within an image. Alternatively or additionally, the computer processor may be configured to automatically derive the position and/or orientation of the tool with respect to the incision region by analyzing an image of the tool and the incision region even without using the tool-identification component.

Reference is now made to FIG. 12, which is a schematic illustration of tool 21 inserted through a patient's cornea 150, such that a tip 170 of the tool is moved in a desired manner within the patient's eye, while entry of the tool into the patient's eye is maintained within an incision zone 172, in accordance with some applications of the present invention. As described hereinabove, the robotic unit is configured to insert tools 21 into the patient's eye such that entry of the tool into the patient's eye is via an incision point, and the tip of the tool is disposed within the patient's eye. For some applications, the robotic unit is configured to move the tip of the tool within the patient's eye in such a manner that entry of the tool into the patient's eye is constrained to remain within the incision point. Alternatively, as shown in FIG. 12, rather than constraining entry of the tool into the patient's eye to remain within an incision point (which is typically only slightly larger than the maximum cross section of the tool that passes through the incision point), entry of the tool into the patient's eye is constrained to remain within an incision zone, which is larger than an incision point. For some applications, the area of the incision zone is more than 150 percent or more than 200 percent of the maximum cross section of the tool that passes through the incision zone. For example, the entry of the tool into the patient's eye may be constrained to remain within an incision zone having an area of 2 mm\hat{ }2 to 10 mm\hat{ }2. Typically, robotic arms 120 are configured to control yaw and pitch angular rotations of the tool, such that a tip of the tool is moved in a desired manner within the patient's eye, while entry of the tool into the patient's eye is maintained within the incision zone.

For some applications, the robotic arms 120 are configured to allow entry of the tool into the patient's eye to move within the incision zone, and the computer processor is configured to drive an output unit to provide feedback to the operator that is indicative of a location of the entry of the tool into the patient's eye within the incision zone. For example, the computer processor may generate an output on display(s) 24 that shows the incision zone and the location of entry of the tool within the incision zone. For some applications, as the tool is moved in such a manner that the location of the entry of the tool into the patient's eye is close to the edge of the incision zone, an output, such as a visual or audio alert, is generated.

As described hereinabove, for some applications, the operator provides commands to the robotic units via control-component unit 26 (shown in FIG. 1). Typically, such commands include commands that control the position and/or orientation of tools that are disposed within the robotic units, and/or commands that control actions that are performed by the tools. For some applications, the computer processor is configured to drive the control-component unit to provide haptic feedback to the operator that is indicative of a location of the entry of the tool into the patient's eye within the incision zone. For example, as the tool is moved in such a manner that the entry location of the tool into the patient's eye is closer to the edge of the incision zone, resistance to movement of the control-component unit may be increased, and/or the control-component unit may be vibrated, and/or a different output may be generated.

Although some applications of the present invention are described with reference to cataract surgery, the scope of the present application includes applying the apparatus and methods described herein to other medical procedures, mutatis mutandis. In particular, the apparatus and methods described herein to other medical procedures may be applied to other microsurgical procedures, such as general surgery, orthopedic surgery, gynecological surgery, otolaryngology, neurosurgery, oral and maxillofacial surgery, plastic surgery, podiatric surgery, vascular surgery, and/or pediatric surgery that is performed using microsurgical techniques. For some such applications, the imaging system includes one or more microscopic imaging units.

It is noted that the scope of the present application includes applying the apparatus and methods described herein to intraocular procedures, other than cataract surgery, mutatis mutandis. Such procedures may include collagen crosslinking, endothelial keratoplasty (e.g., DSEK, DMEK, and/or PDEK), DSO (descemet stripping without transplantation), laser assisted keratoplasty, keratoplasty, LASIK/PRK, SMILE, pterygium, ocular surface cancer treatment, secondary IOL placement (sutured, transconjunctival, etc.), iris repair, IOL reposition, IOL exchange, superficial keratectomy, Minimally Invasive Glaucoma Surgery (MIGS), limbal stem cell transplantation, astigmatic keratotomy, Limbal Relaxing Incisions (LRI), amniotic membrane transplantation (AMT), glaucoma surgery (e.g., trabs, tubes, minimally invasive glaucoma surgery), automated lamellar keratoplasty (ALK), anterior vitrectomy, and/or pars plana anterior vitrectomy.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processor 28) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 28 is typically a hardware device programmed with computer program instructions to produce a special purpose computer. For example, when programmed to perform the algorithms described with reference to the Figures, computer processor 28 typically acts as a special purpose robotic-system computer processor. Typically, the operations described herein that are performed by computer processor 28 transform the physical state of a memory, which is a real physical article, to have a different magnetic polarity, electrical charge, or the like depending on the technology of the memory that is used. For some applications, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for performing robotic microsurgery on a portion of a body of a patient using one or more tools, the apparatus comprising:
at least one robotic unit comprising:
an end effector;
a tool mount coupled to the end effector and configured to securely hold the one or more tools; and
one or more robotic arms coupled to the end effector and which are configured to rotate the one or more tools through pitch and yaw rotations by the one or more robotic arms moving;
at least one XYZ platform configured to move the robotic unit along X and Y directions within an XY plane, and along a Z direction that is perpendicular to the XY plane;
the XYZ platform comprising:
a first slidable shutter that is configured to cover an interior of the XYZ platform by sliding along the X direction as the robotic unit is moved along the X direction; and
a second slidable shutter that is configured to cover an interior of the XYZ platform by sliding along the Y direction as the robotic unit moved along the Y direction.

2. The apparatus according to claim 1, wherein the first slidable shutter is disposed upon and perpendicularly with respect to the second slidable shutter.

3. The apparatus according to claim 1, wherein the XYZ platform comprises a column, wherein the robotic unit is supported upon the column and the column is configured to extend or retract along the Z direction in order to move the robotic unit along the Z direction.

4. The apparatus according to claim 1, wherein each of the slidable shutters is sized such as to provide a range of motion to the robotic unit in each of the X and Y directions of more than 100 mm.

5. The apparatus according to claim 1, wherein:
the at least one robotic unit comprises two robotic units,
the at least one XYZ platform comprises two XYZ platforms, with a respective robotic unit being disposed on each of the XYZ platforms,
a first one of the XYZ platforms and robotic units is configured to be placed on a first side of the portion of the patient's body, and
a second one of the XYZ platforms and robotic units is configured to be placed on a second side of the portion of the patient's body.

6. The apparatus according to claim 1, wherein each of the slidable shutters is configured such that even when the robotic unit is at an extremity of its motion range in either of the X or Y directions, the slidable shutters continue to cover the interior of the XYZ platform.

7. The apparatus according to claim 1, wherein each of the slidable shutters has a length that is greater than a motion range of the robotic unit along a corresponding direction, and has excess shutter length that is configured to roll into an interior of the XYZ platform.

8. The apparatus according to claim 1, further comprising a rotational axis,
wherein the XYZ platform is rotationally coupled to the rotational axis and is configured to:
automatically rotate about the rotational axis from a first position to a second position such as to provide access to the patient to an operator; and
automatically rotate about the rotational axis from the second position to the first position such as to perform the robotic microsurgery on the portion of the patient's body.

9. The apparatus according to claim 8, wherein:
the at least one robotic unit comprises two robotic units,
the at least one XYZ platform comprises two XYZ platforms, with a respective robotic unit being disposed on each of the XYZ platforms,
a first one of the XYZ platforms and robotic units is configured to be placed on a first side of the portion of the patient's body, and
a second one of the XYZ platforms and robotic units is configured to be placed on a second side of the portion of the patient's body.

10. The apparatus according to claim 9, wherein each of the XYZ platforms is configured to:
automatically rotate about the rotational axis from a first position to a second position such as to provide access to the patient to an operator; and
automatically rotate about the rotational axis from the second position to the first position such as to perform the robotic microsurgery on the portion of the patient's body.

11. Apparatus for performing robotic microsurgery on a portion of a body of a patient using one or more tools, the apparatus comprising:
an end effector;
a tool mount coupled to the end effector and configured to securely hold the one or more tools;
a pair of parallel robotic arms coupled to the end effector and which are configured to rotate the one or more tools through a pitch angular rotation by the pair of parallel robotic arms undergoing a change in pitch; and
a pitch-rotation mechanism comprising:
a worm gear coupled to the pair of parallel robotic arms, such that rotation of the worm gear causes the parallel robotic arms to undergo the change in pitch;
a worm screw that engages the worm gear and is configured to rotate the worm gear; and
a spring configured to bias the pair of parallel robotic arms in a given direction, such as to reduce backlash of the parallel robotic arms in response to changes in rotational motion of the worm gear.

12. The apparatus according to claim 11, wherein the pitch-rotation mechanism is configured such that, over a full range of pitch rotation of the one or more tools, a relationship between rotations of the worm screw and an angular pitch rotation of the one or more tools is constant.

13. The apparatus according to claim 11, wherein the pitch-rotation mechanism is configured to move the one or more tools through a pitch angular rotation of at least 60 degrees.

14. The apparatus according to claim 11, wherein the parallel arms are configured to constrain movement of the end effector, and thereby constrain the motion of a tool that is within the tool mount, such that as the tool undergoes changes in pitch, a remote center of motion of the tool is maintained.

15. The apparatus according to claim 14, wherein the tool is configured to be inserted into an eye of the patient via an incision point, and wherein the parallel arms are configured to constrain movement of the end effector, and thereby constrain the motion of the tool that is within the tool mount, such that as the tool undergoes changes in pitch, the remote center of motion of the tool is maintained within the incision point.

16. The apparatus according to claim 14, wherein the tool is configured to be inserted into an eye of the patient via an incision zone, and wherein the parallel arms are configured to constrain movement of the end effector, and thereby constrain the motion of the tool that is within the tool mount, such that as the tool undergoes changes in pitch, the remote center of motion of the tool is maintained within the incision zone.

17-25. (canceled)

26. Apparatus for performing robotic microsurgery on an eye of a patient, the apparatus comprising:
a plurality of tools having different shapes from each other;
an end effector;
a tool mount coupled to the end effector and configured to securely hold the one or more tools;
one or more robotic arms coupled to the end effector and which are configured to move each of the tools while it is held by the tool mount by the one or more robotic arms moving the end effector; and
a computer processor configured to:
drive the robotic arms to insert the tool into the patient's eye through an incision region;
receive an input that is indicative of which tool is being held by the tool mount;
receive a further input indicating that the tool should be retracted from the patient's eye; and
in response to receiving the further input indicating that the tool should be retracted from the patient's eye, move the tool in such a manner that the tool is removed via the incision region.

27. The apparatus according to claim 26, wherein the computer processor is configured to determine a pattern of movement via which to move the tool such that the tool is removed via the incision region, and to retract the tool in accordance with the determine pattern of movement.

28. The apparatus according to claim 26, wherein the computer processor is further configured to determine a position and/or orientation of the tool with respect to the incision region, and to account for the position and/or orientation of the tool with respect to the incision region such as to remove the tool via the incision region.

29-31. (canceled)
